(19) United States
(12) Patent Application Publication
(10) Pub. No.: US 2024/0307014 A1
(43) Pub. Date: Sep. 19, 2024

(54) MEDICAL IMAGE DIAGNOSTIC APPARATUS, MEDICAL IMAGE DIAGNOSTIC METHOD, AND STORAGE MEDIUM

(71) Applicant: CANON MEDICAL SYSTEMS CORPORATION, Otawara-shi (JP)

(72) Inventors: Atsushi FUKANO, Otawara (JP); Kusuto KOGA, Utsunomiya (JP); Terutada SHUTO, Otawara (JP); Masami OGURA, Sano (JP)

(73) Assignee: CANON MEDICAL SYSTEMS CORPORATION, Otawara-shi (JP)

(21) Appl. No.: 18/604,684

(22) Filed: Mar. 14, 2024

(30) Foreign Application Priority Data
Mar. 17, 2023 (JP) 2023-043416

(51) Int. Cl. A61B 6/50; A61B 5/352; A61B 5/353; A61B 6/03; A61B 6/40

(52) U.S. Cl. CPC A61B 6/503; A61B 5/352; A61B 5/353; A61B 6/03; A61B 6/40

(57) ABSTRACT

A medical image diagnostic apparatus of an embodiment has processing circuitry. The processing circuitry is configured to acquire electrocardiographic waveform data of a subject, and set one imaging condition to be applied to electrocardiogram-gated imaging of the subject among a plurality of preset imaging conditions for electrocardiogram-gated imaging on the basis of the acquired electrocardiographic waveform data.

### CROSS-REFERENCE TO RELATED APPLICATION

The present application claims priority based on Japanese Patent Application No. 2023-043416 filed Mar. 17, 2023, the content of which is incorporated herein by reference.

### FIELD

Embodiments disclosed in this specification and the drawings relate to a medical image diagnostic apparatus, a medical image diagnostic method, and a storage medium.

### BACKGROUND

Electrocardiogram-gated imaging is known, in which a heartbeat pattern is predicted on the basis of electrocardiographic waveforms of a subject and a medical image that is less affected by the heartbeat is reconstructed. For example, in electrocardiogram-gated imaging using an X-ray computed tomography (CT) apparatus, imaging conditions are set depending on the heartbeat of a subject from results of breathing exercises of the subject using an electrocardiograph performed before imaging. In accordance with these imaging conditions, the X-ray output of the X-ray CT apparatus is controlled such that imaging at a designated phase can be performed on the basis of a trigger signal (R wave) input from the electrocardiograph during imaging. As a set of imaging conditions, one set of imaging conditions is set according to results of a breathing exercise, and the like, and after the conditions are determined, control is performed under the set imaging conditions.

Electrocardiographic waveforms of a subject are not always stable. Furthermore, for example, for a subject with heart disease, it may be better to perform control on the basis of conditions different from the predetermined trigger signal (R wave). Additionally, the electrocardiographic waveforms may change significantly between the time of a breathing exercise and the time of imaging, making it impossible to collect intended data and requiring re-imaging (re-contrast if a contrast agent is used), which may increase the burden on the subject. A function of monitoring electrocardiographic waveforms of a subject and shifting to continuous exposure if pulse fluctuations or arrhythmia occurs is known. However, it is not possible to respond to the behavior of the heartbeat of a subject with heart disease, and it is impossible to flexibly change imaging conditions in real time in accordance with the heartbeat state of the subject.

### BRIEF DESCRIPTION OF THE DRAWINGS

FIG. 1 is a diagram showing an example of an X-ray CT apparatus 1 according to an embodiment.

FIG. 2 is a diagram showing the relationship between an electrocardiographic waveform of a subject and an exposure phase in electrocardiogram-gated imaging.

FIG. 3 is a diagram showing an example of electrocardiogram-gated imaging conditions C according to an embodiment.

FIG. 4A is a diagram showing the relationship between an electrocardiographic waveform of a subject and an exposure phase under electrocardiogram-gated imaging condition C (condition 1) according to an embodiment.

FIG. 4B is a diagram showing the relationship between the electrocardiographic waveform of the subject and the exposure phase under electrocardiogram-gated imaging condition C (condition 2) according to an embodiment.

FIG. 4C is a diagram showing the relationship between the electrocardiographic waveform of the subject and the exposure phase under electrocardiogram-gated imaging condition C (condition 3) according to an embodiment.

FIG. 4D is a diagram showing the relationship between the electrocardiographic waveform of the subject and the exposure phase under electrocardiogram-gated imaging condition C (condition 4) according to an embodiment.

FIG. 5 is a flowchart showing an example of electrocardiogram-gated imaging processing performed by the X-ray CT apparatus 1 according to an embodiment.

FIG. 6 is a diagram showing an example of an electrocardiogram-gated imaging condition setting screen P1 according to an embodiment.

FIG. 7 is a diagram showing an example of a monitoring screen P2 according to an embodiment.

### DETAILED DESCRIPTION

Hereinafter, a medical image diagnostic apparatus, a medical image diagnostic method, and a storage medium according to embodiments will be described with reference to the drawings. Examples of medical image diagnostic apparatuses include an X-ray CT apparatus, a positron emission tomography (PET)-CT apparatus, a magnetic resonance (MR) apparatus, a general X-ray imaging apparatus, an ultrasound image diagnostic apparatus, a nuclear medicine diagnostic apparatus, and the like. Hereinafter, an example of a case in which the medical image diagnostic apparatus is an X-ray CT apparatus will be described.

A medical image diagnostic apparatus of an embodiment has processing circuitry. The processing circuitry is configured to acquire electrocardiographic waveform data of a subject and set one imaging condition to be applied to electrocardiogram-gated imaging of the subject within a plurality of preset imaging conditions for electrocardiogram-gated imaging on the basis of the acquired electrocardiographic waveform data.

### [Configuration of X-Ray CT Apparatus]

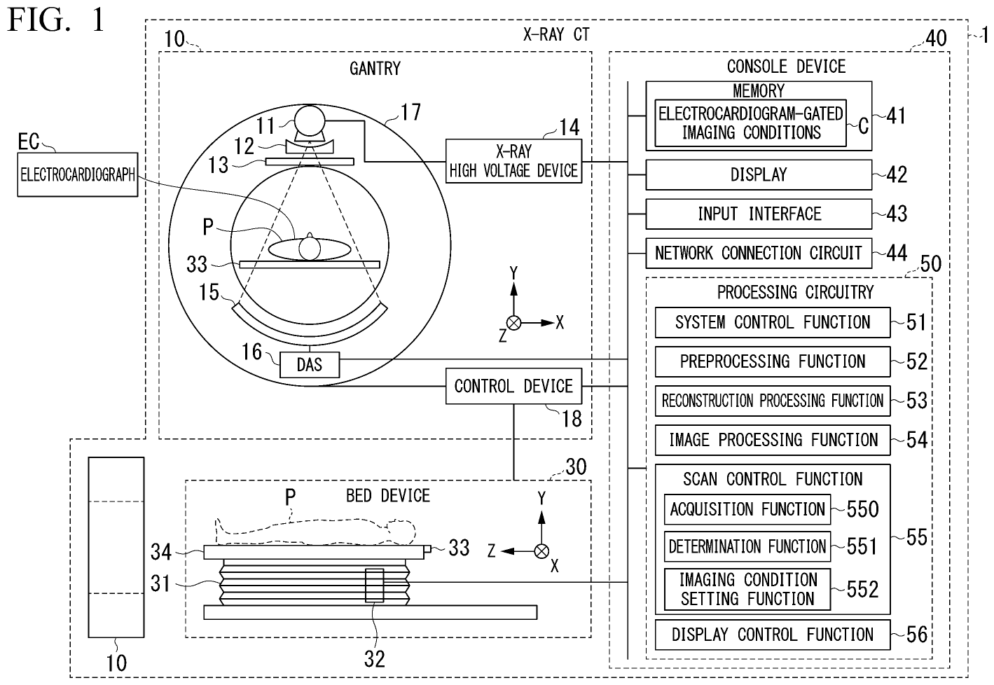

FIG. 1 is a diagram showing an example of an X-ray CT apparatus 1 according to an embodiment. The X-ray CT apparatus 1 has an electrocardiogram-gated imaging function. Electrocardiogram-gated imaging is an imaging technique of predicting a heartbeat pattern of a subject P by acquiring an electrocardiogram (electrocardiographic waveforms) of the subject P and reconstructing an image that is less affected by the heartbeat (an image in which the heart appears to have stopped moving). In the case of electrocardiogram-gated imaging, X-rays are radiated only at the moment when the heart appears to have stopped moving (at a specified respiration phase) to obtain images or X-rays are radiated over time to extract only images captured at the moment when the heart stops for a specified number of heartbeats, and then such data are connected to reconstruct an image.

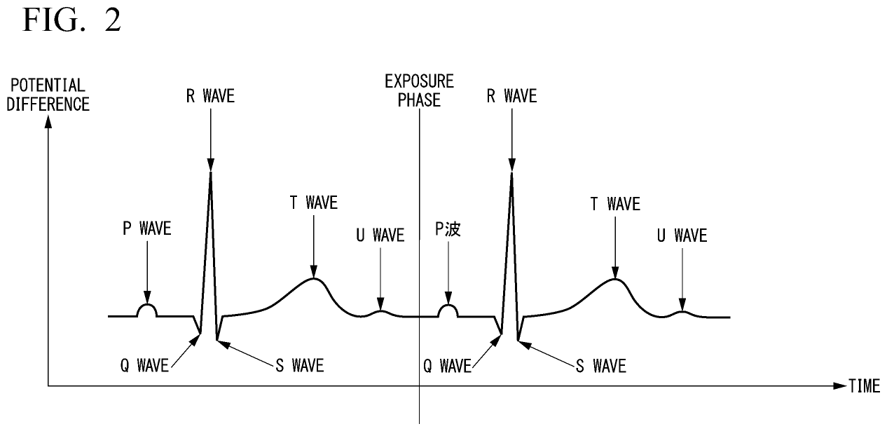

FIG. 2 is a diagram showing a relationship between an electrocardiographic waveform of a subject P and an exposure phase in electrocardiogram-gated imaging. As shown in FIG. 2, the electrocardiographic waveform of a healthy subject P generally includes a fundamental waveform composed of a P wave, a Q wave, an R wave, an S wave, a T wave, and a U wave. When electrocardiogram-gated imaging is performed on the healthy subject P, the R wave, which is the largest wave (apex or peak) indicating the excitement process of the ventricle, is used as a trigger signal. Specifically, during a breathing exercise of the subject P, the time from the peak of an R wave to the peak of the next R wave (hereinafter referred to as “R-R time”) is calculated, and within this R-R time, a phase (for example, a phase around 75% of the R-R time) that is less affected by the heartbeat (the heart appears to have stopped) is set as an exposure phase. Subsequently, at the time of imaging the subject P, the exposure phase is identified on the basis of the timing at which the R wave (trigger signal) is detected, and X-rays are radiated to perform imaging at the identified exposure phase.

As described above, an operator of the X-ray CT apparatus 1 needs to carefully set imaging conditions regarding an exposure phase and other imaging parameters such as a scan rotation speed. In the X-ray CT apparatus 1 of the present embodiment, an imaging condition is appropriately selected according to the state of the heartbeat of the subject P from among a plurality of preset imaging conditions for electrocardiogram-gated imaging, and imaging can be performed in real time under the selected imaging condition.

The X-ray CT apparatus 1 includes, for example, a gantry 10, a bed device 30, and a console device 40. Although both a view of the gantry 10 as viewed in the Z-axis direction and a view as viewed in the X-axis direction are shown in FIG. 1 for convenience of description, in reality, there is only one gantry 10. In the present embodiment, the rotation axis of a rotating frame 17 in a non-tilted state or the longitudinal direction of a top plate 33 of the bed device 30 is defined as the Z-axis direction, an axis perpendicular to the Z-axis direction and horizontal to the floor surface is defined as the X-axis direction, and a direction perpendicular to the Z-axis direction and orthogonal to the floor surface is defined as the Y-axis direction.

The gantry 10 includes, for example, an X-ray tube 11, a wedge 12, a collimator 13, an X-ray high voltage device 14, an X-ray detector 15, a data acquisition system (hereinafter referred to as a DAS) 16, the rotating frame 17, and a control device 18.

The X-ray tube 11 generates X-rays by radiating thermoelectrons from the cathode (filament) toward the anode (target) according to a high voltage applied from the X-ray high voltage device 14. The X-ray tube 11 includes a vacuum tube. For example, the X-ray tube 11 is a rotating anode type X-ray tube that generates X-rays by radiating thermoelectrons to a rotating anode.

The wedge 12 is a filter for adjusting an X-ray dose radiated from the X-ray tube 11 to the subject P. The wedge 12 attenuates X-rays that pass through the wedge 12 such that the distribution of X-ray dose radiated from the X-ray tube 11 to the subject P becomes a predetermined distribution. The wedge 12 is also called a wedge filter or a bow-tie filter. The wedge 12 is, for example, made of aluminum processed to have a predetermined target angle and a predetermined thickness.

The collimator 13 is a mechanism for narrowing down a radiation range of X-rays that have passed through the wedge 12. The collimator 13 narrows down the radiation range of X-rays by forming a slit using a combination of a plurality of lead plates, for example. The collimator 13 is sometimes called an X-ray diaphragm. The narrowing range of the collimator 13 may be mechanically drivable.

The X-ray high voltage device 14 includes, for example, a high voltage generator (not shown) and an X-ray control device (not shown). The high voltage generator has an electric circuit including a transformer, a rectifier, and the like and generates a high voltage to be applied to the X-ray tube 11. The X-ray control device controls the output voltage of the high voltage generator according to an X-ray dose to be generated in the X-ray tube 11. The high voltage generator may be one that boosts a voltage using the above-mentioned transformer or may boost a voltage using an inverter. The X-ray high voltage device 14 may be provided on the rotating frame 17 or may be provided on a fixed frame (not shown) side of the gantry 10.

The X-ray detector 15 detects the intensity of X-rays generated by the X-ray tube 11 and incident through the subject P. The X-ray detector 15 outputs an electrical signal (an optical signal or the like) corresponding to the detected intensity of X-rays to the DAS 16. The X-ray detector 15 has, for example, a plurality of X-ray detection element rows. Each of the plurality of X-ray detection element rows has a plurality of X-ray detection elements arranged in a channel direction along an arc having the focal point of the X-ray tube 11 as a center. The plurality of X-ray detection element rows are arranged in a slice direction (column direction or row direction).

The X-ray detector 15 is, for example, an indirect detector that includes a grid, a scintillator array, and an optical sensor array. The scintillator array has a plurality of scintillators. Each scintillator has a scintillator crystal. A scintillator crystal emits light in an amount corresponding to the intensity of incident X-rays. The grid is disposed on the surface of the scintillator array on which X-rays are incident and has an X-ray shielding plate that has a function of absorbing scattered X-rays. Note that the grid is sometimes called a collimator (one-dimensional collimator or two-dimensional collimator). The optical sensor array includes optical sensors such as photomultiplier tubes (PMTs), for example. The optical sensor array outputs an electrical signal according to the amount of light emitted by the scintillator. The X-ray detector 15 may be a direct conversion type detector having a semiconductor element that converts incident X-rays into electrical signals.

The DAS 16 includes, for example, an amplifier, an integrator, and an A/D converter. The amplifier performs amplification processing on an electrical signal output by each X-ray detection element of the X-ray detector 15. The integrator integrates the amplified electrical signal over a view period (which will be described later). The A/D converter converts the electrical signal representing the integration result into a digital signal. The DAS 16 outputs detection data based on the digital signal to the console device 40. The detection data is a digital value of the x-ray intensity identified by a channel number and a row number of an X-ray detection element that is a generation source, and a view number indicating a collected view. The view number is a number that changes according to rotation of the rotating frame 17, and is a number that is incremented according to rotation of the rotating frame 17, for example. Therefore, the view number is information indicating the rotation angle of the X-ray tube 11. A view period is a period that falls between a rotation angle corresponding to a certain view number and a rotation angle corresponding to the next view number. The DAS 16 may detect switching of views using a timing signal input from the control device 18, an internal timer, or a signal obtained from a sensor that is not shown. In the case of full scanning, when X-rays are continuously exposed by the X-ray tube 11, the DAS 16 collects a group of detection data for the entire circumference (360 degrees). In the case of half scanning, when X-rays are continuously exposed by the X-ray tube 11, the DAS 16 collects detection data for half the circumference (180 degrees).

The rotating frame 17 is an annular member that supports the X-ray tube 11, the wedge 12, the collimator 13, and the X-ray detector 15 in a facing manner. The rotating frame 17 is rotatably supported by a fixed frame around the subject P introduced therein. The rotating frame 17 further supports the DAS 16. Detection data output by the DAS 16 is transmitted via optical communication from a transmitter having a light emitting diode (LED) provided in the rotating frame 17 to a receiver having a photodiode provided in a non-rotating part (for example, a fixed frame) of the gantry 10 and transferred by the receiver to the console device 40. Note that the method of transmitting detection data from the rotating frame 17 to the non-rotating part is not limited to the method using optical communication described above, and any contactless transmission method may be employed. The rotating frame 17 is not limited to an annular member, and may be an arm-like member as long as it can support and rotate the X-ray tube 11 and the like.

Although the X-ray CT apparatus 1 is, for example, a rotate/rotate-type X-ray CT apparatus (third generation CT) in which both the X-ray tube 11 and the X-ray detector 15 are supported by the rotating frame 17 and rotate around the subject P, it is not limited thereto and may be a stationary/rotate-type X-ray CT apparatus (fourth generation CT) in which a plurality of X-ray detection elements arranged in an annular shape are fixed to a fixed frame and the X-ray tube 11 rotates around the subject P.

The control device 18 includes, for example, processing circuitry including a processor such as a central processing unit (CPU). The control device 18 receives an input signal from an input interface attached to the console device 40 or the gantry 10 and controls the operations of the gantry 10 and the bed device 30. The control device 18 ascertains the rotation angle of the rotating frame 17 from the output of a sensor that is not shown, or the like. Further, the control device 18 provides the rotation angle of the rotating frame 17 to a scan control function 55 at any time. The control device 18 may be provided on the gantry 10 or may be provided on the console device 40.

The bed device 30 is a device on which the subject P to be scanned is placed and moved, and is introduced into the rotating frame 17 of the gantry 10. The bed device 30 includes, for example, a base 31, a bed driving device 32, the top plate 33, and a support frame 34. The base 31 includes a housing that supports the support frame 34 movably in the vertical direction (Y-axis direction). The bed driving device 32 includes a motor and an actuator. The bed driving device 32 moves the top plate 33 along the support frame 34 in the longitudinal direction of the top plate 33 (Z-axis direction). Further, the bed driving device 32 moves the top plate 33 in the vertical direction (Y-axis direction). The top plate 33 is a plate-shaped member on which the subject P is placed.

The bed driving device 32 may move not only the top plate 33 but also the support frame 34 in the longitudinal direction of the top plate 33. Further, contrary to the above, the gantry 10 may be movable in the Z-axis direction, and the rotating frame 17 may be controlled to come around the subject P by moving the gantry 10. Alternatively, both the gantry 10 and the top plate 33 may be movable. Further, the X-ray CT apparatus 1 may be an apparatus in which the subject P is scanned in a standing or sitting position. In this case, the X-ray CT apparatus 1 includes a subject support mechanism in place of the bed device 30, and the gantry 10 rotates the rotating frame 17 about an axial direction perpendicular to the floor surface.

The console device 40 includes, for example, a memory 41, a display 42, an input interface 43, a network connection circuit 44, and processing circuitry 50. Although the console device 40 is described as being separate from the gantry 10 in the present embodiment, the gantry 10 may include some or all of the components of the console device 40.

The memory 41 is realized by, for example, a random access memory (RAM), a semiconductor memory element such as a flash memory, a hard disk, an optical disk, or the like. The memory 41 stores, for example, electrocardiogram-gated imaging conditions C, detection data, projection data, reconstructed image data, CT image data, information regarding the subject P, imaging conditions, and the like. Such data may be stored in an external memory with which the X-ray CT apparatus 1 can communicate instead of the memory 41 (or in addition to the memory 41). The external memory is controlled by a cloud server, for example, when the cloud server that manages the external memory receives a read/write request.

### (Electrocardiogram-Gated Imaging Conditions)

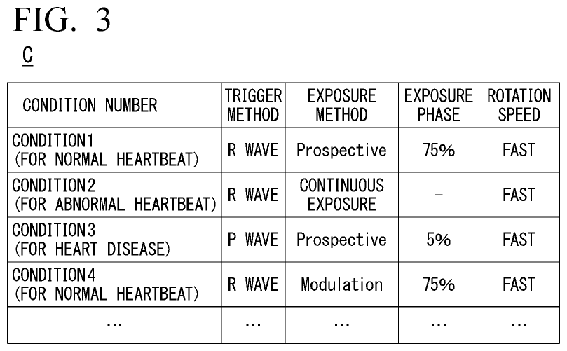

FIG. 3 is a diagram showing an example of electrocardiogram-gated imaging conditions C according to an embodiment. As shown in FIG. 3, the electrocardiogram-gated imaging conditions C include a plurality of different conditions (e.g., conditions 1 to 4). The electrocardiogram-gated imaging conditions C may be set in advance when the system is introduced, or may be set on the basis of an input operation performed by the operator via the input interface 43. In the electrocardiogram-gated imaging conditions C, the content of an imaging condition is associated with a condition number that identifies each condition. The imaging conditions include, for example, a trigger method, an exposure method, an exposure phase, a rotation speed, and the like. The imaging conditions may include other imaging conditions.

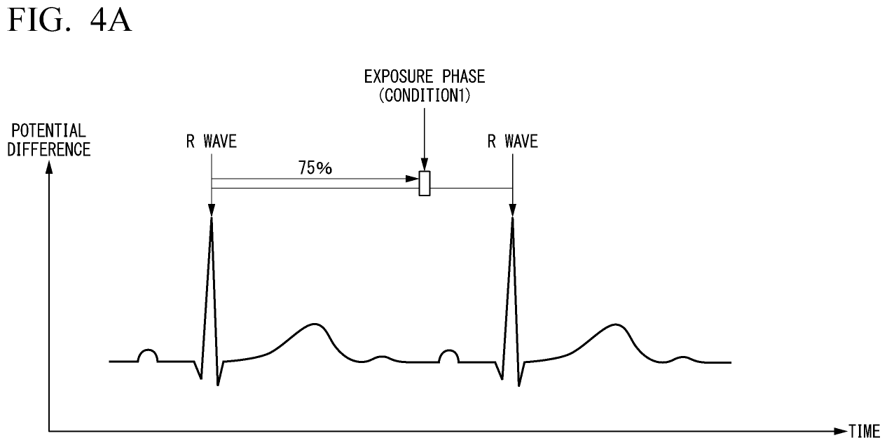

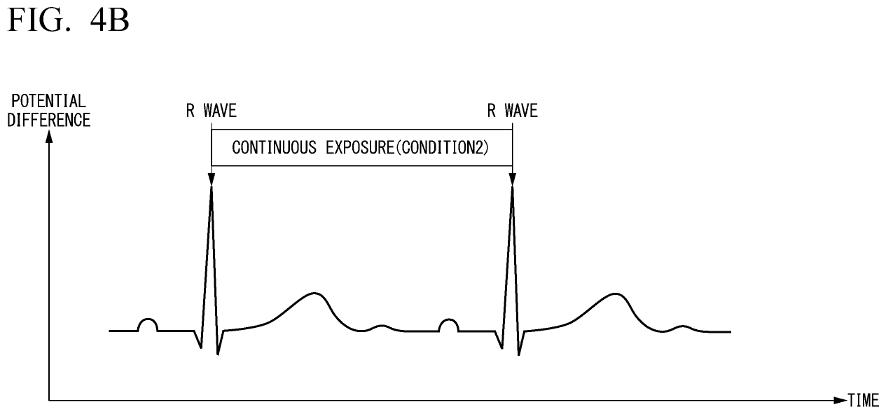

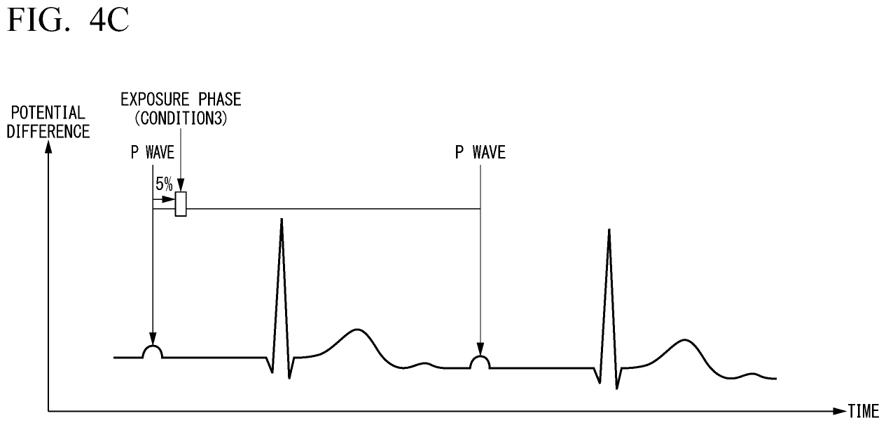

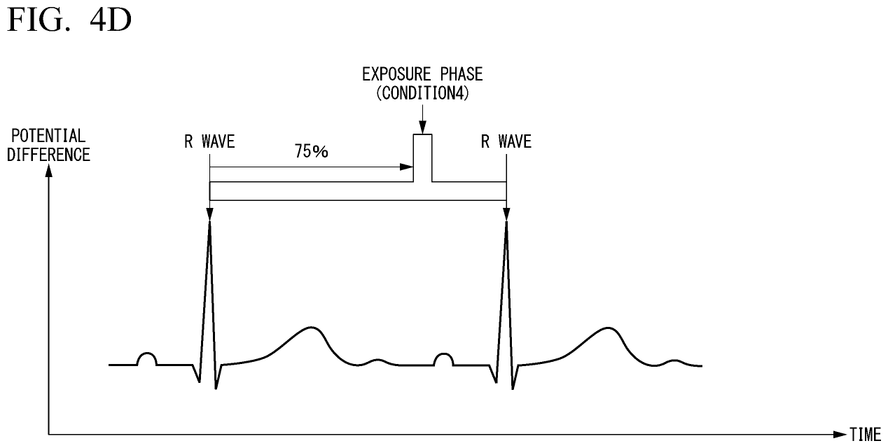

Condition 1 (for normal heartbeat) is an imaging condition for healthy subjects. Condition 1 is that the trigger method is “R wave,” the exposure method is “prospective,” the exposure phase is “75%,” and the rotation speed is “fast.” FIG. 4A is a diagram showing the relationship between an electrocardiographic waveform of a subject and an exposure phase in this electrocardiogram-gated imaging condition C (condition 1). As shown in FIG. 4A, in condition 1, the “R wave” is set as the trigger method (trigger signal), and the exposure phase is set to “75%” phase of the R-R time. Since the exposure method is “prospective,” X-ray exposure is selectively performed in this 75% phase (or in a predetermined phase range including the 75% phase) when the rotation speed of the rotating frame 17 is “fast.”

Condition 2 (for abnormal heartbeat) is an imaging condition for subjects with abnormal heartbeat. For example, condition 2 is used (i) cases where the heartbeat is not stable during breathing exercises (a case where arrhythmia continues and a case where an electrocardiographic waveform has no periodicity), (ii) cases where a normal heartbeat can be confirmed during breathing exercises and “condition 1 (for normal heartbeat)” is set as the initial condition, but the heartbeat becomes abnormal during subsequent imaging, and (iii) cases where a normal heartbeat can be confirmed during breathing exercises and “condition 1 (for normal heartbeat)” is set as the initial condition, but the behavior of the heartbeat (the length between R and R) during subsequent imaging is different from that during breathing exercises. Condition 2 is that the trigger method is “R wave,” the exposure method is “continuous exposure,” the exposure phase is “—(no setting),” and the rotation speed is “fast.” FIG. 4B is a diagram showing the relationship between an electrocardiographic waveform of a subject and an exposure phase in this electrocardiogram-gated imaging condition C (condition 2). As shown in FIG. 4B, in condition 2, the “R wave” is set as the trigger method (trigger signal), and continuous exposure is performed during the R-R time when the rotation speed of the rotating frame 17 is set to “fast.”

Condition 3 (for heart disease) is an imaging condition for subjects with heart disease (atrioventricular (AV) block, or the like). Condition 3 is used in a case where the waveform of heart disease is confirmed as a result of breathing exercises, and the like. For example, compared to a healthy subject, the waveform of a subject with heart disease may have a longer period between a P wave and an R wave. In such a case, the “P wave” is used instead of the “R wave” as a trigger signal. Condition 3 is that the trigger method is the “P wave,” the exposure method is “prospective,” the exposure phase is “5%”, and the rotation speed is “fast.” FIG. 4C is a diagram showing the relationship between an electrocardiographic waveform of a subject and an exposure phase in this electrocardiogram-gated imaging condition C (condition 3). As shown in FIG. 4C, in condition 3, the “P wave” is set as the trigger method (trigger signal), and the exposure phase is set to “5%” phase between a P wave and the next P wave (hereinafter referred to as “P-P time”). Since the exposure method is “prospective,” X-ray exposure is selectively performed in this 5% phase (or in a phase region of a predetermined range including the 5% phase) when the rotation speed of the rotating frame 17 is “fast.” In this manner, by performing X-ray exposure (imaging) immediately after the P wave, it becomes possible to perform electrocardiogram-gated imaging with high precision even on a subject with heart disease. Note that the imaging condition for a subject with heart disease can be arbitrarily set depending on the state of the heartbeat of the subject.

Condition 4 (for normal heartbeat) is an imaging condition for healthy subjects. Condition 4 is that the trigger method is the “R wave,” the exposure method is “modulation,” the exposure phase is “75%,” and the rotation speed is “fast.” FIG. 4D is a diagram showing the relationship between an electrocardiographic waveform of a subject and an exposure phase under this electrocardiogram-gated imaging condition C (condition 4). As shown in FIG. 4D, under condition 4, the “R wave” is set as the trigger method (trigger signal), and the exposure phase is set to “75%” phase of the R-R time. Since the exposure method is “modulation,” during the R-R time, selective high-dose X-ray exposure in the 75% phase and continuous low-dose X-ray exposure in a phase range other than the 75% phase are performed when the rotation speed of the rotating frame 17 is “fast.”

That is, the imaging conditions include a plurality of modes for defining a specific period (exposure phase) regarding X-ray exposure. In the first mode (condition 1) of the plurality of modes, the specific period is set on the basis of the R wave in electrocardiographic waveform data, and in the second mode (condition 3) of the plurality of modes, the specific period is set on the basis of the P wave in electrocardiographic waveform data. In the second mode, the specific period is set such that it falls between the P wave and the R wave on the basis of the P wave. Further, the specific period is a period in which X-rays are radiated, or a period in which a higher dose of X-rays is radiated than in periods other than the specific period.

That is, the imaging conditions include at least waveform information (trigger signal) that serves as a reference for a timing of imaging (exposure phase) in electrocardiogram-gated imaging. Furthermore, the imaging conditions further include a timing of imaging (exposure phase) and an imaging method (exposure method) in electrocardiogram-gated imaging. The plurality of imaging conditions include at least a first condition (conditions 1 and 4) for subjects with normal heartbeat, a second condition (condition 2) for subjects with abnormal heartbeat, and a third condition (condition 3) for subjects with heart disease. The trigger signal in the first condition and the trigger signal in the third condition are different from each other. The trigger signal in the first condition is an R wave in electrocardiographic waveform data, and the trigger signal in the third condition is a P wave in electrocardiographic waveform data.

The display 42 displays various types of information. For example, the display 42 displays a medical image (CT image) generated by the processing circuitry, a graphical user interface (GUI) image for receiving various operations of an operator such as a doctor or a technician, and the like. The display 42 is, for example, a liquid crystal display, a cathode ray tube (CRT), an organic electroluminescence (EL) display, or the like. The display 42 may be provided on the gantry 10. The display 42 may be of a desktop type, or may be a display device (for example, a tablet terminal) that can communicate wirelessly with the main body of the console device 40. The display 42 is an example of a “display.”

The input interface 43 receives various input operations performed by the operator, and outputs an electrical signal indicating the content of a received input operation to the processing circuitry 50. For example, the input interface 43 receives input operations such as electrocardiogram-gated imaging conditions, collection conditions at the time of collecting detection data or projection data, reconstruction conditions at the time of reconstructing a CT image, and image processing conditions at the time of generating a post-processed image from a CT image. For example, the input interface 43 is realized by a mouse, a keyboard, a touch panel, a track ball, a switch, a button, a joystick, a camera, an infrared sensor, a microphone, or the like. The input interface 43 may be provided in the gantry 10. Further, the input interface 43 may be realized by a display device (for example, a tablet terminal) that can communicate wirelessly with the main body of the console device 40. Note that in this specification, the input interface is not limited to one that includes physical operation parts such as a mouse and a keyboard. For example, examples of the input interface include electrical signal processing circuitry that receives an electrical signal corresponding to an input operation from an external input device provided separately from the device and outputs this electrical signal to a control circuit.

The network connection circuit 44 includes, for example, a network card having a printed circuit board, a wireless communication module, and the like. The network connection circuit 44 implements an information communication protocol depending on the type of network to be connected.

The processing circuitry 50 controls the overall operation of the X-ray CT apparatus 1, the operation of the gantry 10, and the operation of the bed device 30. The processing circuitry 50 executes, for example, a system control function 51, a preprocessing function 52, a reconstruction processing function 53, an image processing function 54, a scan control function 55, a display control function 56, and the like. These components are realized, for example, by a hardware processor (computer) executing a program (software) stored in the memory 41. The hardware processor means, for example, circuitry such as a CPU, a graphics processing unit (GPU), an application specific integrated circuit (ASIC), a programmable logic device (for example, a simple programmable logic device (SPLD) or a complex programmable logic devices (CPLDs)), or a field programmable gate array (FPGA). Instead of storing the program in the memory 41, the program may be directly incorporated into the circuit of the hardware processor. In this case, the hardware processor realizes the functions by reading and executing the program incorporated into the circuit. The hardware processor is not limited to being configured as a single circuit, but may be configured as one hardware processor by combining a plurality of independent circuits to realize each function. Further, a plurality of components may be integrated into one hardware processor to realize each function.

Each component included in the console device 40 or the processing circuitry 50 may be distributed and realized by a plurality of pieces of hardware. The processing circuitry 50 may be realized by a processing device that can communicate with the console device 40 instead of the components included in the console device 40. The processing device is, for example, a workstation connected to one X-ray CT apparatus, or a device (e.g., a cloud server) that is connected to a plurality of X-ray CT apparatuses and collectively executes the same processing as the processing circuitry 50 which will be described below.

The system control function 51 controls various functions of the processing circuitry 50 on the basis of input operations received through the input interface 43.

The preprocessing function 52 performs preprocessing such as logarithmic conversion processing, offset correction processing, inter-channel sensitivity correction processing, and beam hardening correction on detection data output by the DAS 16 to generate projection data.

The reconstruction processing function 53 performs reconstruction processing on projection data generated by the preprocessing function 52 using a filtered back projection method, a successive approximation reconstruction method, or the like to generate CT image data and stores the generated CT image data in the memory 41.

On the basis of an input operation received through the input interface 43, the image processing function 54 converts CT image data into three-dimensional image data or cross-sectional image data of an arbitrary cross section using a known method. Conversion to three-dimensional image data may be performed by the preprocessing function 52.

The scan control function 55 controls detection data collection processing in the gantry 10 by instructing the X-ray high voltage device 14, the DAS 16, the control device 18, and the bed driving device 32. The scan control function 55 controls the operation of each part when capturing a positioning image and when capturing an image used for diagnosis.

The scan control function 55 includes, for example, an acquisition function 550, a determination function 551, and an imaging condition setting function 552. The acquisition function 550 acquires electrocardiographic waveform data of the subject P measured by an electrocardiograph EC during breathing exercises, imaging, and the like. The acquisition function 550 may acquire diagnostic information of the subject P from other medical image diagnostic apparatuses (modality such as an ultrasound diagnostic apparatus), a picture archiving and communication system (PACS), an electronic medical record server, and the like, which are not shown and store the diagnostic information in the memory 41 or a radiology information system (RIS). The acquisition function 550 is an example of an “acquisition unit.”

The determination function 551 determines a waveform pattern (type) of the acquired electrocardiographic waveform data of the subject P. This waveform pattern includes, for example, a normal waveform pattern indicating that the heartbeat is normal, an abnormal waveform pattern indicating that the heartbeat is abnormal, a heart disease waveform pattern indicating heart disease, and the like. The determination function 551 determines a waveform pattern on the basis of periodicity of each waveform (P wave, R wave, and the like) included in the electrocardiographic waveform data of the subject P, for example. For example, when the R wave has periodicity (when the R-R time can be considered constant), the determination function 551 determines that the waveform pattern is a normal waveform pattern. Further, the determination function 551 determines that the waveform pattern is an abnormal waveform pattern, for example, when the R wave has no periodicity (when the R-R time is not constant). Further, the determination function 551 determines that the waveform pattern is a heart disease waveform pattern, for example, when the interval tendency of each waveform is different from that of a healthy subject (for example, when a P-R time is long).

The imaging condition setting function 552 sets one imaging condition to be applied to electrocardiogram-gated imaging for the subject P from among a plurality of preset imaging conditions for electrocardiogram-gated imaging on the basis of the acquired electrocardiographic waveform data or the determined pattern. The imaging condition setting function 552 automatically sets one imaging condition or sets one imaging condition on the basis of an input instruction from the operator via the input interface 43. The imaging condition setting function 552 is an example of an “imaging condition setting unit.”

The display control function 56 causes the display 42 to display various images (captured images, positioning images, input operation results received by the input interface 43, and the like). The display control function 56 causes the display 42 to display, for example, an electrocardiogram-gated imaging condition setting screen, a monitoring screen, and the like. The display control function 56 is an example of a “display control unit.”

With the above configuration, the X-ray CT apparatus 1 scans the subject P in a scanning manner such as helical scanning, conventional scanning, or step-and-shoot. Helical scanning is a mode in which the subject P is scanned in a spiral manner by rotating the rotating frame 17 while moving the top plate 33. Conventional scanning is a mode in which the rotating frame 17 is rotated while the top plate 33 is kept stationary to scan the subject P in a circular orbit. The step-and-shoot is a mode in which the position of the top plate 33 is moved at regular intervals to perform conventional scanning in a plurality of scan areas.

### [Processing Flow]

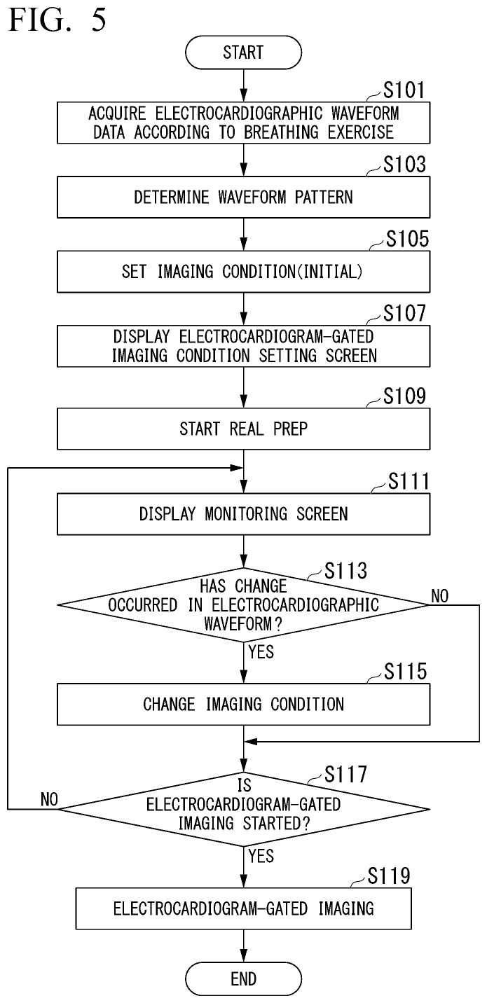

Next, electrocardiogram-gated imaging processing performed by the X-ray CT apparatus 1 according to the present embodiment will be described. FIG. 5 is a flowchart showing an example of the electrocardiogram-gated imaging processing performed by the X-ray CT apparatus 1 according to an embodiment. In this flowchart, an example of processing of proceeding from real prep (contrast agent monitoring scan) to electrocardiogram-gated imaging will be described. It is assumed that the memory 41 of the console device 40 stores electrocardiogram-gated imaging conditions C in advance. Further, it is assumed that the subject P to be scanned is placed on the bed device 30 and positioning scan has been completed.

First, the subject P, who is the subject of electrocardiogram-gated imaging, practices breathing. During this breathing exercise, the electrocardiograph EC attached to the subject P measures electrocardiographic waveform data. The acquisition function 550 acquires electrocardiographic waveform data of the subject P from the electrocardiograph EC (step S101).

Next, the determination function 551 determines a waveform pattern of the acquired electrocardiographic waveform data of the subject P (step S103). This waveform pattern includes, for example, a normal waveform pattern indicating that the heartbeat is normal, an abnormal waveform pattern indicating that the heartbeat is abnormal, and a heart disease waveform pattern indicating heart disease.

Next, the imaging condition setting function 552 sets one imaging condition (initial) to be applied to electrocardiogram-gated imaging for the subject P among a plurality of imaging conditions included in the electrocardiogram-gated imaging conditions C on the basis of the acquired electrocardiographic waveform data or the determined waveform pattern (step S105). The imaging condition setting function 552 sets one imaging condition automatically or on the basis of an input instruction from the operator via the input interface 43.

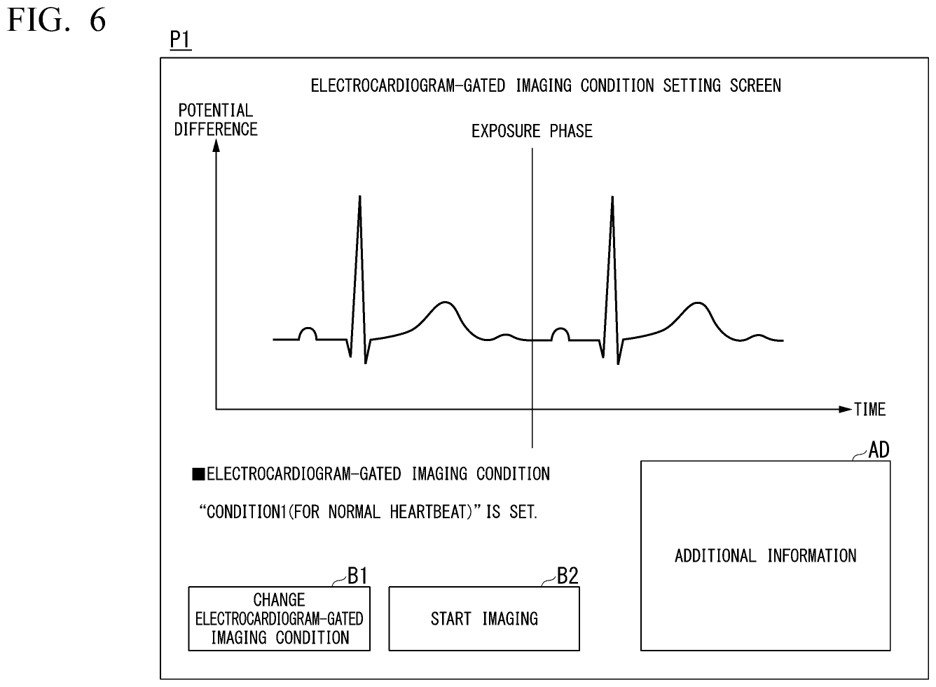

Next, the display control function 56 causes the display 42 to display the electrocardiogram-gated imaging condition setting screen (step S107). FIG. 6 is a diagram showing an example of the electrocardiogram-gated imaging condition setting screen P1 according to an embodiment. As shown in FIG. 6, the electrocardiogram-gated imaging condition setting screen P1 display an exposure phase according to the set imaging conditions on the electrocardiographic waveform data measured by the electrocardiograph EC. Here, condition 1 (for normal heartbeat) is set as the imaging condition, the trigger method is “R wave,” the exposure method is “prospective,” and the exposure phase is “75%.” Furthermore, the electrocardiogram-gated imaging condition setting screen P1 displays a button B1 for receiving an instruction to change imaging conditions and a button B2 for receiving an instruction to start imaging. Further, the electrocardiogram-gated imaging condition setting screen P1 displays, as additional information AD, diagnostic information of the subject P measured by other modalities, for example. Accordingly, the operator can determine imaging conditions for electrocardiogram-gated imaging to be performed thereafter.

That is, the display control function 56 causes the display 42 (display) to display the electrocardiographic waveform data and diagnostic information (additional information AD) measured by an apparatus different from the medical image diagnostic apparatus.

Next, the scan control function 55 starts real prep (step S109). As a result, administration of a contrast medium to the subject P is started, and monitoring of spread of the contrast medium within the subject P and the electrocardiographic waveform of the subject P measured by the electrocardiograph EC is started. Furthermore, the display control function 56 causes the monitoring screen to be displayed on the display 42 (step S111).

During monitoring, the acquisition function 550 continuously acquires electrocardiographic waveform data of the subject P from the electrocardiograph EC, and the determination function 551 determines whether a change has occurred in the waveform pattern of the acquired electrocardiographic waveform data of the subject P (step S113).

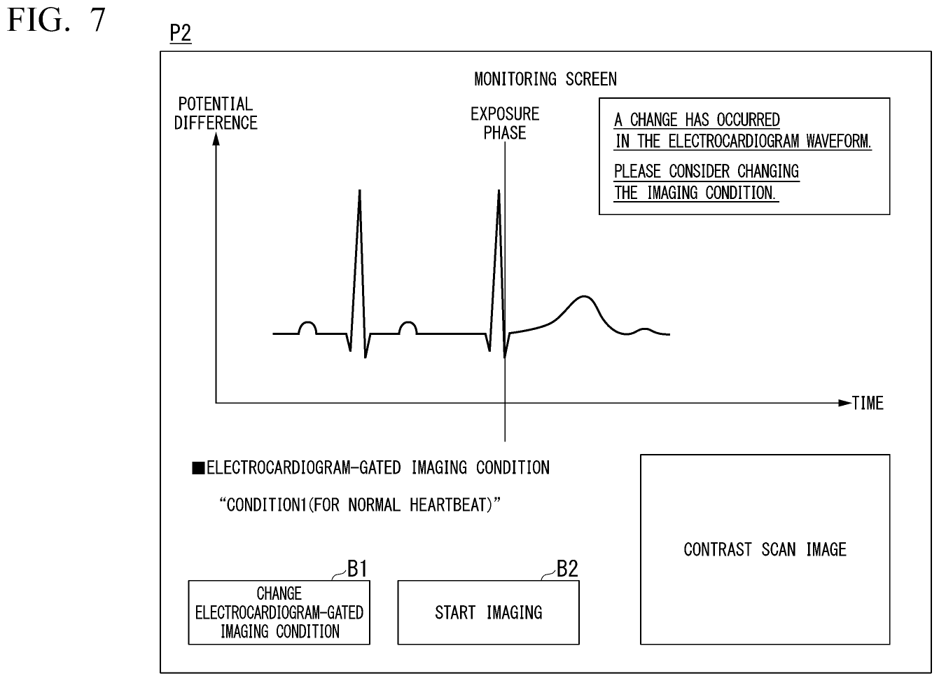

If the determination function 551 determines that a change has occurred in the waveform pattern (step S113; YES), the imaging condition setting function 552 changes one imaging condition applied to electrocardiogram-gated imaging for the subject P on the basis of the acquired electrocardiographic waveform data or the waveform pattern determined to have a change (step S115). Furthermore, the display control function 56 may display, on the display 42, a notification that a change has occurred in the waveform pattern on the monitoring screen. FIG. 7 is a diagram showing an example of the monitoring screen P2 according to an embodiment. As shown in FIG. 7, on the monitoring screen P2, a message notifying that a change has occurred in electrocardiographic waveform data is displayed along with a contrast agent scan image and electrocardiographic waveform data measured by the electrocardiograph EC. In the example of FIG. 7, a message is displayed saying, “A change has occurred in the electrocardiographic waveform. Please consider changing the imaging condition.” Accordingly, the operator who is monitoring the electrocardiographic waveform can ascertain the occurrence of a change in the waveform pattern not only by his own determination but also by notification from the system. This allows the operator to manually change the imaging condition by inputting an instruction via the input interface 43. That is, the display control function 56 causes the display 42 to display a notification that a change has occurred in the electrocardiographic waveform data during contrast agent monitoring scan of the subject.

For example, in a case where condition 1 (for normal heartbeat) is set as the initial condition of imaging conditions in step S105 above, and there is a change in the electrocardiographic waveform in the subsequent real prep, the imaging condition setting function 552 changes the imaging condition to condition 2 (for abnormal heartbeat) or condition 3 (for heart disease) automatically or on the basis of an input instruction from the operator via the input interface 43, for example.

Alternatively, in a case where condition 3 (for heart disease) is set as the initial imaging condition in step S105 above, and there is a change in the electrocardiographic waveform in the subsequent real prep, the imaging condition setting function 552 changes the imaging condition to condition 1 (for normal heartbeat) or condition 2 (for abnormal heartbeat) automatically or on the basis of an input instruction from the operator via the input interface 43, for example.

That is, the acquisition function 550 acquires electrocardiographic waveform data of the subject measured at the first timing (during breathing exercise) before performing electrocardiogram-gated imaging, and the imaging condition setting function 552 sets one imaging condition to be applied to electrocardiogram-gated imaging performed thereafter on the basis of the electrocardiographic waveform data measured at the first timing. In addition, the acquisition function 550 further acquires electrocardiographic waveform data of the subject measured at a second timing (at the time of contrast agent monitoring scan in real prep) after the first timing before electrocardiogram-gated imaging is performed, and the imaging condition setting function 552 changes one imaging condition on the basis of the electrocardiographic waveform data measured at the second timing.

Note that before the imaging condition is automatically changed by the imaging condition setting function 552, a screen for receiving consent from the operator regarding the change may be displayed.

After the determination function 551 determines that there is no change in the waveform pattern (step S113; NO) or after the imaging condition setting function 552 changes the imaging condition (step S115), the scan control function 55 determines whether conditions for starting electrocardiogram-gated imaging are satisfied on the basis of spread of a contrast agent in the subject P, and the like and determines whether or not to start electrocardiogram-gated imaging (step S117). If the scan control function 55 determines to start electrocardiogram-gated imaging (step S117; YES), electrocardiogram-gated imaging is performed (step S119). On the other hand, if the scan control function 55 determines that electrocardiogram-gated imaging is not started (step S117; NO), the display control function 56 continues displaying the monitoring screen (step S111) and repeats the subsequent processing. Accordingly, processing of this flowchart ends.

According to the present embodiment described above, stable electrocardiogram-gated imaging can be performed under imaging conditions depending on the state of the heartbeat of a subject. For example, an initial imaging condition can be set during breathing exercises in advance, and during subsequent imaging, an optimal imaging condition can be automatically set or the imaging condition can be changed in real time by the operator, and thus both exposure reduction and stable scanning can be achieved. For example, when imaging a subject with heart disease, an imaging condition for heart disease is set as the initial imaging condition, and if the heart rate becomes normal during subsequent imaging, the imaging condition can be changed to an imaging condition for normal heartbeat. In addition, in a case where the imaging condition for normal heartbeat is set as the initial imaging condition and the heartbeat becomes abnormal during subsequent imaging, the imaging condition can be changed to an imaging condition for abnormal heartbeat (for example, continuous exposure) to avoid re-imaging. In addition, in a case where the imaging condition for abnormal heartbeat (e.g., continuous exposure) is set as the initial imaging condition and the heartbeat becomes normal during subsequent imaging, the imaging condition can be changed to the imaging condition for normal heartbeat (e.g., prospective) to reduce the exposure of the subject.

Note that, if diagnostic information of a subject acquired in advance (for example, diagnostic information measured by an apparatus different from the medical image diagnostic apparatus) indicates that the subject has heart disease, the imaging condition setting function 552 may set the imaging condition for subjects with heart disease as an initial condition of one imaging condition by default, and if electrocardiographic data of the subject shows a normal heartbeat during contrast agent monitoring scan of the subject, change the imaging condition to an imaging condition for subjects with a normal heartbeat.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image diagnostic apparatus comprising processing circuitry configured to:
acquire electrocardiographic waveform data of a subject; and
set one imaging condition to be applied to electrocardiogram-gated imaging of the subject among a plurality of preset imaging conditions for electrocardiogram-gated imaging on the basis of the acquired electrocardiogram waveform data.

2. The medical image diagnostic apparatus according to claim 1, wherein the imaging condition includes waveform information serving as a reference for imaging timing in the electrocardiogram-gated imaging, imaging timing in the electrocardiogram-gated imaging, and an imaging method.

3. The medical image diagnostic apparatus according to claim 1, wherein the plurality of imaging conditions include at least a first condition for a subject with a normal heartbeat, a second condition for a subject with an abnormal heartbeat, and a third condition for a subject with heart disease.

4. The medical image diagnostic apparatus according to claim 1, wherein the processing circuitry is configured to:
acquire electrocardiographic waveform data of the subject measured at a first timing before the electrocardiogram-gated imaging is performed; and
set the one imaging condition to be applied to the electrocardiogram-gated imaging to be performed thereafter on the basis of the electrocardiographic waveform data measured at the first timing.

5. The medical image diagnostic apparatus according to claim 4, wherein the processing circuitry is further configured to:
acquire electrocardiographic waveform data of the subject measured at a second timing after the first timing before the electrocardiogram-gated imaging is performed; and
change the one imaging condition on the basis of the electrocardiographic waveform data measured at the second timing.

6. The medical image diagnostic apparatus according to claim 5, wherein
the first timing is during breathing exercise, and
the second timing is during a contrast agent monitoring scan.

7. The medical image diagnostic apparatus according to claim 1, wherein the processing circuitry is configured to automatically set the one imaging condition, or set the one imaging condition on the basis of an instruction from an operator.

8. The medical image diagnostic apparatus according to claim 1, wherein
the medical image diagnostic apparatus is an X-ray CT apparatus,
the plurality of imaging conditions include a plurality of modes for defining a specific period regarding X-ray radiation, and
the processing circuitry is configured to:
set the specific period on the basis of an R wave in the electrocardiographic waveform data in a first mode among the plurality of modes; and
set the specific period on the basis of a P wave in the electrocardiographic waveform data in a second mode among the plurality of modes.

9. The medical image diagnostic apparatus according to claim 8, wherein the processing circuitry is configured to set the specific period on the basis of the P wave to make the specific period fall between the P wave and the R wave in the second mode.

10. The medical image diagnostic apparatus according to claim 8, wherein the specified period is a period in which X-rays are radiated or a period in which a higher dose of X-rays is radiated than in periods other than the specified period.

11. The medical image diagnostic apparatus according to claim 1, wherein
the medical image diagnostic apparatus is an X-ray CT apparatus, and
the imaging condition includes at least an exposure phase of X-rays, an exposure method, and a trigger signal indicating a waveform serving as a reference for the exposure phase in the electrocardiogram-gated imaging.

12. The medical image diagnostic apparatus according to claim 11, wherein
the plurality of imaging conditions include at least a first condition for a subject with a normal heartbeat, a second condition for a subject with an abnormal heartbeat, and a third condition for a subject with heart disease, and
the trigger signal in the first condition and the trigger signal in the third condition are different from each other.

13. The medical image diagnostic apparatus according to claim 12, wherein
the trigger signal in the first condition is an R wave in the electrocardiographic waveform data, and
the trigger signal in the third condition is a P wave in the electrocardiographic waveform data.

14. The medical image diagnostic apparatus according to claim 1, wherein the processing circuitry is further configured to cause a display to display the electrocardiographic waveform data and diagnostic information measured by an apparatus different from the medical image diagnostic apparatus.

15. The medical image diagnostic apparatus according to claim 1, wherein the processing circuitry is configured to:
set an imaging condition for a subject with heart disease as an initial condition of the one imaging condition in a case where diagnostic information of the subject obtained in advance indicates that the subject has heart disease; and
change the imaging condition to an imaging condition for a subject with a normal heartbeat in a case where the electrocardiographic waveform data of the subject indicates a normal heartbeat during a contrast agent monitoring scan of the subject.

16. The medical image diagnostic apparatus according to claim 1, wherein the processing circuitry is further configured to cause the display to display a notification that a change has occurred in the electrocardiographic waveform data during a contrast agent monitoring scan of the subject.

17. A medical image diagnostic method using a computer of a medical image diagnostic apparatus, comprising:
acquiring electrocardiographic waveform data of a subject; and
setting one imaging condition to be applied to electrocardiogram-gated imaging of the subject among a plurality of preset imaging conditions for electrocardiogram-gated imaging on the basis of the acquired electrocardiographic waveform data.

18. A computer-readable non-transitory storage medium storing a program causing a computer of a medical image diagnostic apparatus to:
acquire electrocardiographic waveform data of a subject; and
set one imaging condition to be applied to electrocardiogram-gated imaging of the subject among a plurality of preset imaging conditions for electrocardiogram-gated imaging on the basis of the acquired electrocardiographic waveform data.
